(19) United States
(12) Patent Application Publication
(10) Pub. No.: US 2025/0268664 A1
(43) Pub. Date: Aug. 28, 2025

(54) SURGERY DEVICE AND METHOD FOR ADJUSTING A MEDICAL APPARATUS

(71) Applicant: Erbe Elektromedizin GmbH, Tuebingen (DE)

(72) Inventors: Faisal KALIM, Reutlingen (DE); Ovidiu JURJUT, Bensheim (DE); Shirish JOSHI, Wurmlingen (DE)

(21) Appl. No.: 19/057,291

(22) Filed: Feb. 19, 2025

(30) Foreign Application Priority Data
Feb. 22, 2024 (EP) 24159116.3

(51) Int. Cl. A61B 34/00; A61B 34/20; A61B 90/00

(52) U.S. Cl. CPC A61B 34/25; A61B 90/361; A61B 2034/2065; A61B 2034/2068; A61B 2090/373

(57) ABSTRACT

The device includes an image capturing device to which an image processing device and via the latter an image display device is connected. The image processing device is configured to show a menu in the image of image display device. Showing the menu can be triggered by a suitable movement of instrument. For example, an activation range can be provided for this purpose at the image border that is monitored by the image processing device for determining whether the instrument tip touches this region. If so, a respective activation signal characterizing this gesture can be created and can be used for showing menu in the image. Additional gestures of the instrument tip, particularly in a defined detection region, allow the control of a pointer in the menu and the selection of functions for control of apparatus.

### CROSS REFERENCE TO RELATED APPLICATION

This application claims the benefit of European Patent Application No. 24159116.3, filed Feb. 22, 2024, which is incorporated herein by reference in its entirety.

### TECHNICAL FIELD

The invention refers to a device for treatment of the body of a human or animal patient. Particularly, the invention refers to a device in which an apparatus supplying the treating instrument can be adjusted by means of manipulation of the instrument as well as a method for this purpose.

### BACKGROUND

A system, a method and a device for following an instrument by means of a digital microscope is known from WO 2022/216810 A2. It creates an image and allows the operation of a graphical user interface depicted in the image by means of the tip of the instrument that can be recognized by an image analysis device. The graphical user interface shown in the image is preferably depicted in the edge area of the image outside of the operation region of interest. If required, the user interface can also be blanked out. As soon as the user moves the tip of the instrument into the area of the graphical user interface, the latter can be shown. Subsequently, respective fields of the graphical user interface can be selected in that the instrument tip resides in this area for a minimum duration.

For operation of the graphical user interface, the user has to move the instrument tip into the graphical user interface located at the image border and has to leave the instrument tip there for the duration of the operation.

### SUMMARY

It is an object of the invention to provide a device that allows an alternative control operation of a graphical user interface by means of an instrument. Further, it is an object of the invention to provide a method for control operation of the graphical user interface.

These objects are solved by means of the devices and methods described herein.

The device according to the invention comprises an image capturing device that is configured to capture an image of the operation site and to transmit it to an image processing device. The image capturing device can be an endoscope camera or another camera. Alternatively, an endoscope can be provided having one or more working channels and a camera at its distal end for capturing images or a video stream of an operation site.

Additionally, an instrument is part of the device, the instrument tip of which can be moved into the field of view of the image capturing device. For example, such an instrument can be an instrument that is separate from the image capturing device and can be moved independently from the latter. It can also be a rigid or flexible instrument configured as a probe that can be moved into and positioned inside the field of view of the image capturing device. A positioning device can serve for positioning of the instrument tip inside the field of view, wherein by means of the positioning device the instrument tip can be moved transverse to its longitudinal direction. For this purpose, means can be provided in or on the instrument by means of which the instrument tip can be bent. If the instrument is a laparoscopic instrument having a rigid shank, the handle provided at the proximal end is the positioning means by means of which the distal end can be positioned.

Connected to the image processing device is an image display device, which is configured to display the image captured by the image capturing device. For example, the image display device can be a monitor, VR glasses or the like. The image display device can be arranged in mediate proximity of the operation site or, for example, in case of remote operation, also in larger distance thereto.

The image display device and the image processing device are configured to show a menu in the represented image if required, that represents a graphical user interface. Preferably, this menu is arranged outside the center of the captured image in order not to cover the interesting region and the region subject to surgical operation of human or animal tissue that is typically located in the center of the captured image. The menu shown outside the center contains a pointer symbol. Preferably, it is always present. Further preferably, the pointer symbol is only movable within the menu, but not beyond its edge.

The image processing device is configured to detect the instrument, particularly its instrument tip, and to reproduce movements of the instrument tip with the pointer symbol. The pointer symbol and the instrument tip are thus movably coupled. Thereby, the pointer symbol is represented with distance to the instrument tip. In doing so, the instrument tip can remain in the image center, that means in the area of the operation site, during a control operation of the menu, that means the graphical user interface, nevertheless, the instrument tip can be used for control of the menu. This is particularly advantageous if a longer movement of the instrument tip shall be avoided from a surgical point of view or if the region of the shown menu is only difficult to reach. In a preferred embodiment the image display device and/or the image processing device are configured to define a detection region in the image located outside of the menu. Moreover, the image display device and/or the image processing device can be configured to use the instrument tip and its movements only for moving and positioning the pointer if the instrument tip is located in the detection region.

Preferably, an edge zone is defined for the image visible on the image display device, wherein the image display device and/or the image processing device monitors whether the instrument tip has been positioned in the edge zone. The image display device and/or the image processing device can be configured to show the usually invisible menu in the image represented on the image display device as soon as the instrument tip is positioned in the edge zone and/or remains there for longer duration.

After showing the menu, a pointer symbol that is visible inside the menu can be moved by means of the instrument tip in order to select individual menu fields. For this purpose, the image processing device can be particularly configured to move the pointer symbol according to the movement of the instrument tip as soon as a movement of the instrument tip is detected by means of the image processing device. Thereby, the image processing device can be configured to use a movement of the instrument tip for moving the pointer symbol only if the instrument tip is positioned inside the detection region provided for this purpose, that is, for example, arranged centrally. The detection region can be preferably arranged in the center of the image inside which the instrument tip is located anyway when carrying out a surgical operation.

The individual menu fields can be assigned to different technical actions, such as particularly the selection or change of settings of a generator or another apparatus supplying the instrument. For example, the menu fields can be assigned to different modes and/or different effect strengths. The menu can contain submenus, for example in order to be able to change the effect strengths in selected modes.

The movement of the instrument tip can be transferred into the movement of the pointer in proportional manner (that means with constant distance to the pointer symbol) as well as—if required—using a magnification factor or a reduction factor by means of which the path length the pointer travels compared with the path length the instrument tip travels is increased or decreased respectively. In doing so, the path length that the pointer moves during a movement of the instrument tip can be longer or shorter than the path length of the instrument tip movement. However, in any case, it is preferred that the directions of the paths of the instrument tip and the pointer coincide with each other in the image at any point in time.

The device according to the invention further comprises an acknowledging device that serves to select or activate that menu field in which the pointer is present. The acknowledging device can be a foot switch, a manual switch or also a gesture recognition device that can be part of the image processing device. For example, a gesture serving for triggering an acknowledging signal can be a piercing movement of the instrument tip. Also, a circular movement of the instrument tip can be detected as acknowledging signal. Also, other gestures for generating an acknowledging signal can be provided. For example, a selection of menu fields can be carried out by means of a vertical movement of the pointer and a confirmation (that is acknowledgement) of a menu field can be carried out by means of a horizontal movement of the pointer inside the menu field.

In a preferred embodiment an activation signal is created for showing a menu, in that an activation region is touched with the instrument tip. The control operation of the menu by means of the instrument tip is preferably carried out only if the latter is located inside this detection region. An acknowledgement signal for confirmation of a selected menu field can be created, if the instrument tip carries out a predefined gesture inside the detection region, for example a piercing movement.

A facilitated control operation of a generator by the surgeon is possible with the device according to the invention and the respective method according to the invention, without requiring the surgeon to look away from the surgical operation site and without requiring the surgeon to leave the surgical operation site sustainably.

### BRIEF DESCRIPTION OF THE DRAWINGS

Additional details and advantages of an embodiment according to the invention as well as modifications thereof are derived from the following description and the drawing. The drawing shows:

FIG. 1 the device according to the invention in an overview illustration,

FIGS. 2 to 7 an image represented by an image display device and the tip of the instrument visible therein in different control operation conditions in schematic illustration.

### DETAILED DESCRIPTION

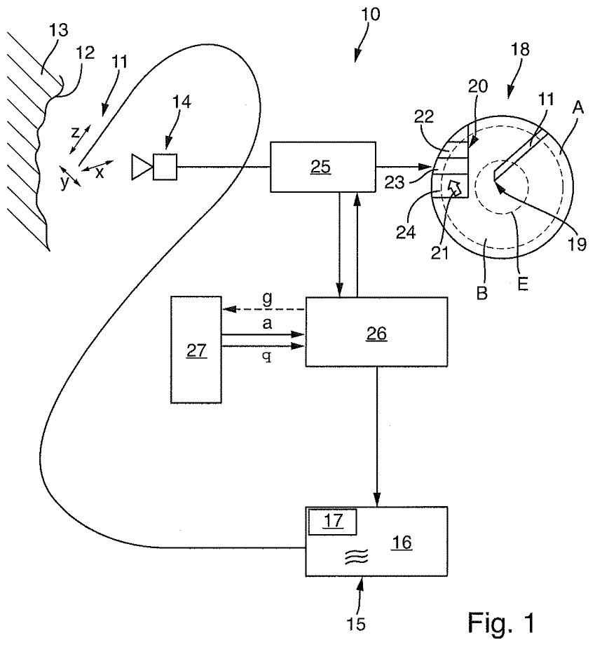

From FIG. 1 a device 10 for surgical treatment of a patient is known, who is illustrated in FIG. 1 only by means of a tissue surface 12 that is to be treated by means of an instrument 11. The tissue surface 12 is an operation site and can be, for example, the interior surface of a hollow organ 13, such as the stomach, the intestine or other organs or tissue regions. In the illustration according to FIG. 1, it is assumed that the surgical operation is carried out endoscopically, wherein the illustration of the endoscope has been omitted. For this reason, only the instrument 11—which can be configured as flexible probe, for example—as well as an image capturing device 14 are illustrated that can be an endoscope camera, for example.

The instrument 11 can be configured in rigid manner, for example as a laparoscopic instrument, or can be configured in flexible manner, for example as an endoscope probe. For carrying out a surgical operation it is brought into interaction or at least in proximity with the tissue surface 12 and is moved according to the requirements of the surgical operation process. Movement directions are symbolized in FIG. 1 by means of arrows that symbolize the movement directions x, y, z. The instrument 11 configured as probe can be guided through a working channel of an endoscope, wherein the endoscope can comprise an image capturing device 14. For example, the instrument 11 can also be configured in rigid manner, wherein a handle for positioning the rigid instrument 11 inside the patient can be provided at the proximal end. During laparoscopic surgical operations the image capturing device 14 can be inserted into the body of the patient separately and independent from instrument 11. All elements that are provided for moving the (flexible or rigid) instrument 11 together form the positioning device for the instrument 11.

For example, the instrument 11 is an RF surgical instrument that is connected for this purpose with a supplying apparatus 15, for example in form of an RF generator 16. However, also other applications are possible. For example, the apparatus 15 can be an apparatus that is configured additionally or alternatively to the RF creation for gas supply of instrument 11, for purging the operation site, for gas extraction or for other purposes.

For adjustment of apparatus 16 the latter can have quite numerous adjustment means 17, such as push buttons, rotational buttons, screens and the like. The control operation thereof, however, requires orientation toward the apparatus 12 either by the surgeon himself/herself that guides the instrument 11 or by another operator person. The attention of the surgeon is, however, preferably orientated to the display device 18 showing the picture B captured by the image capturing device 14. In the latter, the distal end of the instrument 11 with its instrument tip 19 is visible and in the field of view of the image capturing device 14 and thus also movable within the picture B represented by the display device 18.

If required, graphical elements can be shown in picture B, such as a menu 20 and a pointer 21, which is present in the menu 20. The pointer 21 is a graphical symbol, for example, a dot, a circle, a triangle or an arrow or the symbol of a hand or another symbol. The menu can comprise multiple menu fields 22, 23, 24, wherein the pointer 21 is movable over these menu fields 22, 23, 24. The menu fields 22, 23, 24 can be assigned to functions, such as turning functions of the apparatus 15 on and off, influencing effect strengths and/or showing of submenus for additional command input. The menu fields 22, 23, 24 can be rectangular or otherwise formed areas that can be provided with identifying colors, symbols or labels in order to clarify the functions that are selectable via the menu fields. Preferably, the pointer 21 is translucently depicted so that the function of a menu field 22, 23, 24 remains visible and recognizable, if pointer 21 is placed on the respective menu field.

An image processing device 25 is arranged between the image capturing device 14 and the image display device 18, wherein the image processing device 25 is configured to localize the image tip 19 in the image captured by the image capturing device 14 and to control apparatus 15 based thereon. For this purpose, the image processing device 25 is connected with a control device 26 that is configured to create control commands for apparatus 15 and transmit them to the apparatus 15. The control device 26 can be additionally connected with an acknowledging device 27 that can be provided to create acknowledging signals in order to cause control device 26 and image processing device 25 to show the menu 20 within the image. Additionally, the acknowledging device 27 can be configured to create acknowledging signals q in order to activate menu fields 22, 23 or 24 onto which pointer 21 is positioned. Upon receipt of an acknowledging signal q, control device 26 provides a control signal to apparatus 15 corresponding to the concerned menu field 22, 23 or 24.

The acknowledging device 27 can be a manual acknowledging device, for example in form of a push button that can be actuated by hand or foot. Additionally, it can be a device for recognition of gestures of the instrument tip 19. The recognition of such gestures can be carried out by means of the image processing device 25 and/or the control device 26. For example, the image processing device 25 can recognize respective gestures g and transmit corresponding signals to the acknowledging device 27 that thereupon generates corresponding activation signals a or acknowledging signals q and transmits them to the control device 26. Alternatively, the image processing device 25 can be configured to recognize the gestures and to send a corresponding signal g to the acknowledging device 27 or directly to the control device 26. The selection of a menu field, for example, by means of a movement of the pointer 21 along the sequence of menu fields 22, 23, 24, can here be carried out as a vertical movement, for example. A gesture for confirmation (i.e. acknowledgement) of a menu field (for example menu field 23) and for creation of an acknowledging signal q can be a horizontal movement of pointer 21 inside the concerned menu field 23, for example.

The device 10 described so far operates as follows:

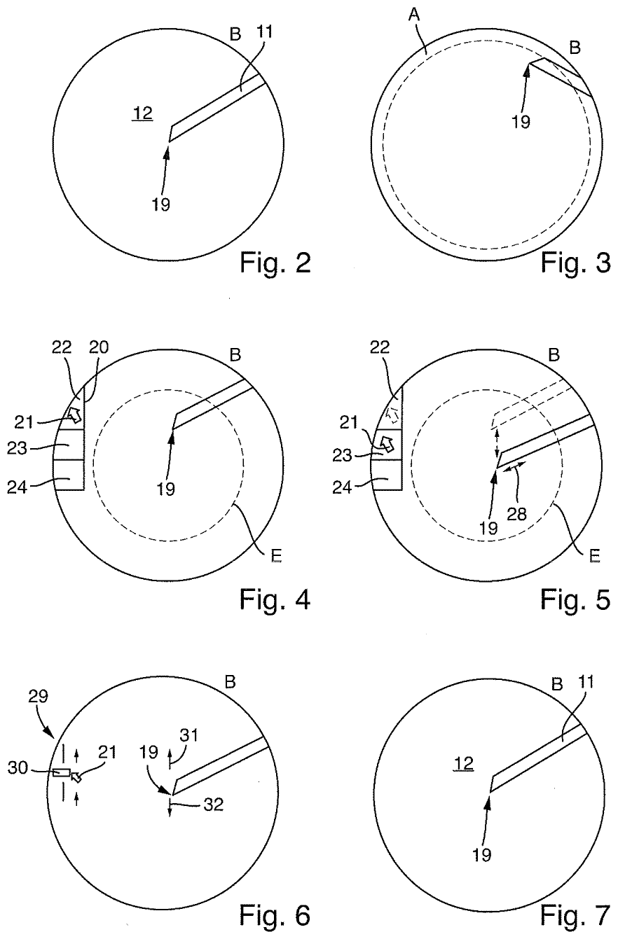

First, FIG. 2 illustrates image B with instrument tip 19 visible therein, captured by image capturing device 14. The instrument tip is approximately depicted in the center of the image and thus in the typically interesting region of the tissue 12 that shall be influenced by means of instrument 11.

It is now assumed that the operator desires to adjust apparatus 15 without looking away from image B. Therefore, he/she moves the instrument tip 19 now into an activation region A that can be, for example, the entire ring-shaped border region of image B. As soon as instrument tip 19 touches this activation region A, as illustrated in FIG. 3, the image processing device 25 or the control device 26 detects this as gesture g and sends a respective signal to the acknowledging device 27. The latter generates an activation signal a that causes the image processing device 25 either on the indirect route via the control device 26 or also on the direct route to show menu 20 within the image B, as visible in FIG. 4.

Upon showing menu 20 in image B according to FIG. 4, a detection region E can be defined that covers a part of image B, for example a central region or that alternatively covers the entire image B. This detection region E is monitored by means of the image processing device 25 for movements of instrument tip 19 as soon as the latter is located inside detection region E. Movements of the instrument tip 19 detected by image processing device 25 are transferred into respective movements of pointer 21.

For example, this is shown based on the correlation of FIG. 4 and FIG. 5. If instrument tip 19 is moved from the position according to FIG. 4 into the position according to FIG. 5, which is illustrated in FIG. 5 by means of a dashed arrow, pointer 21 is moved out of the former position illustrated in dashed lines in FIG. 5 and is moved from menu field 22 into menu field 23. The direction of the movement of pointer 21 thereby always coincides with the direction of the movement of the instrument tip 19. In this manner, pointer 21 can be moved over different menu fields 22, 23, 24 in order to select one of the menu fields 22 to 24. Preferably, the movement range of pointer 21 is limited to the menu 20, that means the pointer 21 is unable to leave menu 20.

The selection of a menu field 22, 23 or 24 is carried out subsequently by means of an acknowledging signal q. The latter can be produced by means of acknowledging device 27 and can be transmitted to control unit 26. A push button, for example on the instrument, a foot switch or also the recognition of a gesture can serve for creation of an acknowledging signal q. According to FIG. 5, for example, a piercing gesture can be carried out with instrument 11, which is indicated in FIG. 5 by means of arrow 28. The piercing gesture can be a short, quick movement of the instrument in instrument longitudinal direction. If this gesture is carried out inside detection region E, it can be recognized and supplied as acknowledging signal q to the control device 26. The latter can thereupon, depending on the meaning of the selected field, here the menu field 21, transfer generator 16 (or another apparatus 15) into the mode assigned to the menu field 23, for example coagulation.

If desired, the image processing device 25 can show an additional menu field 29 in the image B according to FIG. 6, for example in order to be able to adjust the strength of the desired effect. Thereby, pointer 21 can move a slider 30 upward and downward that is symbolically illustrated as graphic, which can be caused by a respective movement of instrument tip 19 in the direction of arrows 31, 32. After a predetermined time has passed or alternatively by means of a respective gesture, for example again the piercing gesture according to arrow 28 in FIG. 5, menu field 29 can be hidden again, whereupon the image according to FIG. 7 is visible with the instrument 11 therein without additional graphical depictions. The instrument 11 is now ready for treatment of the tissue surface 12 with the selected settings.

In the description above it has been assumed that the movement of the instrument tip 19, particularly in vertical direction, is transmitted onto pointer 21 in 1:1 ratio. However, it is also possible to transmit the path along which the instrument tip 19 has been moved in magnified manner on pointer 21, for example in order to be able to control menu 20 with minimum instrument movements. Alternatively, the path of pointer 21 can also be a proportionally reduced version of the path the instrument tip 19 has been moved, for example, in order to allow specifically sensitive menu control operations. Particularly, it is possible to define different transmission ratios from instrument tip 19 to pointer 21 for the main menu 20 and the menu field 29. Additionally, it is possible to use another selected point of instrument 11 for control of menu 20 and pointer 21 instead of the instrument tip 19. The present description of the invention and its embodiments then applies accordingly under the condition that the selected position of the instrument 11 replaces the instrument tip 19.

It is further possible to only transmit vertical movement of instrument tip 19 to the pointer 21 while a horizontal movement of the instrument tip 19 is not transmitted to the pointer 21. In this manner, the control operation of menu 20 is simplified and operating errors are avoided, particularly moving pointer 21 out of menu 20 or sticking of pointer 21 on the edge of the menu. The image processing device 25 is not required to, but can be configured to limit the movability of pointer 21 on the menu 20. Also, this simplifies the control operation and allows particularly to control pointer 21 at the position inside detection range E by means of instrument tip 19, where the instrument 11 can be moved best.

The device 10 according to the invention comprises an image capturing device 14 to which an image processing device 25 and via the latter an image display device 18 are connected. The image processing device 25 is configured to show a menu 20 in the image B of image display device 18. Showing the menu 20 can be triggered by a suitable movement of instrument 11. For example, an activation region A can be provided for this purpose at the image border that is monitored by means of image processing device 25 whether the instrument tip 19 touches this region. If this is the case, a respective activation signal A characterizing this gesture can be created and can be used for showing menu 20 in the image. Additional gestures of instrument tip 19, particularly in a defined detection region E, allow the control of a pointer 21 in the menu 20 and the selection of functions for control of apparatus 15.

### REFERENCE SIGNS

10 device for surgical treatment of a patient
11 instrument
12 tissue surface
13 hollow organ
14 image capturing device
X, y, z movement directions
15 apparatus for supply of instrument 11
16 generator
17 operating element
18 image display device
B image on image display device 18
19 instrument tip
20 menu
21 pointer
22-24 menu fields
25 image processing device
26 control device/detection device
27 acknowledging device
A activation region
E detection region
28 arrow
29 menu field for effect strength adjustment
30, 31 arrows

1. A device (10) for surgical treatment of a human or animal patient, comprising:
an image capturing device (14) configured to capture an image (B) of an operation site (12) and to transmit the image (B) to an image processing device (25);
an instrument (11) configured to influence the patient at the operation site (12) and to be movably guidable within the operation site (12); and
an image display device (18) connected to the image processing device (25), wherein the image display device (18) is configured to;
show the image (B) captured from the operation site (12), and if required:
show at least one menu (20) outside a center of the captured image (B); and
show a pointer symbol (21) that is movably coupled with the instrument (11).

2. The device according to claim 1, wherein the instrument (11) comprises a distal instrument tip (19) that can be positioned in the operation site (12).

3. The device according to claim 2, wherein assigned to the instrument (11) is a positioning device, which is configured to move the distal instrument tip (19) inside the image (B) captured by the image capturing device (14) in two directions (x, y) that are independent from one another.

4. The device according to claim 2, wherein the pointer symbol (21) is movably coupled with the distal instrument tip (19).

5. The device according to claim 4, wherein the pointer symbol (21) is positioned distant from the distal instrument tip (19) in the image (B) in at least one direction (x, y).

6. The device according to claim 1, wherein the at least one menu (20) comprises multiple menu fields (22, 23, 24, 29) to which different technical actions are assigned and the different technical actions are a selection or modification of settings of an apparatus (15) for supply of the instrument (11), the settings being part of different modes and effect strengths.

7. The device according to claim 2, further comprising a detection device (26) connected with the image processing device (25), wherein the detection device (26) is configured to detect the position of the distal instrument tip (19) in the image (B) and to transmit the position of the distal instrument tip (19) to the image processing device (25), wherein the image processing device (25) is configured to move the pointer symbol (21) depending on changes of the position of the distal instrument tip (19).

8. The device according to claim 7, wherein the image processing device (25) is configured to couple a movement of the pointer symbol (21) using a magnification factor or a reduction factor to a movement of the instrument tip (19).

9. The device according to claim 7, further comprising an acknowledging device (27) that is configured to create an acknowledging signal (q) and that is connected with the detection device (26) in order to transmit the acknowledging signal (q) to the detection device (26).

10. The device according to claim 7, wherein the detection device (26) is configured to transmit adjustment parameters to an apparatus (15) for the supply of the instrument (11) upon receipt of an acknowledging signal (q), wherein the adjustment parameters are stored for a menu field (22, 23, 24) on which the pointer symbol (21) is positioned.

11. The device according to claim 9, wherein the image processing device (25) is configured to show and to hide the at least one menu (20) depending on an activation command (a).

12. The device according to claim 11, wherein the acknowledging device (27) is configured to create the activation command (a).

13. The device according to claim 12, wherein the acknowledging device (27) comprises a hand or foot push button.

14. The device according to claim 12, wherein the acknowledging device (27) is configured to detect predefined movements of the distal instrument tip (19) and to create acknowledging signals (q) and/or activation signals (a) depending on the detected predefined movements.

15. A method for adjusting an apparatus (15) for supply of a surgical instrument (11), wherein the method comprises:
capturing an image (B) of an operation site (12) by an image capturing device (14) and transmitting the image (B) to an image processing device (25);
positioning an instrument (11) suitable for influencing a patient at the operation site (12) with an instrument tip (19) thereof at the operation site (12);
displaying the captured image (B) of the operation site (12) and of the instrument tip (19) with an image display device (18);
displaying at least one menu (12) outside of a center of the captured image (B) with the image display device (18); and
displaying a pointer (21) on the at least one menu (20) that is movably coupled with the instrument (11).
